(19) United States
(12) Patent Application Publication
(10) Pub. No.: US 2024/0334045 A1
(43) Pub. Date: Oct. 3, 2024

(54) SYSTEMS AND METHODS FOR STANDARDIZED TEETH WHITENESS MEASUREMENT AND MONITORING TEETH WHITENING PROGRESS USING A PORTABLE ELECTRONIC DEVICE

(71) Applicant: Abdullaibrahim Abdulwaheed, Holbrook, MA (US)

(72) Inventor: Abdullaibrahim Abdulwaheed, Holbrook, MA (US)

(21) Appl. No.: 18/738,498

(22) Filed: Jun. 10, 2024

Related U.S. Application Data
(63) Continuation-in-part of application No. 17/825,489, filed on May 26, 2022, now abandoned.
(63) Continuation-in-part of application No. 17/391,823, filed on Aug. 2, 2021, now abandoned.

(51) Int. Cl. H04N 23/60; A61B 1/24; G06T 7/00; G16H 40/20; H04N 23/611; H04N 23/63; H04N 23/71; H04N 23/74

(52) U.S. Cl. CPC H04N 23/64; A61B 1/24; G06T 7/0016; G16H 40/20; H04N 23/611; H04N 23/635; H04N 23/71; H04N 23/74; G06T 2207/20104; G06T 2207/30036

(57) ABSTRACT

A system and method for standardized teeth whiteness measurement and monitoring teeth whitening progress using a portable electronic device are disclosed. The system includes an application that assists users in aligning their teeth with an on-screen alignment guide. It further ensures standardized lighting conditions for image capture through adjustable illumination zones, adjusted according to ambient lighting and the specific characteristics of the device. Post-alignment, users have the option to manually outline a selected portion of their teeth for whiteness analysis. This manual outline aids in maintaining consistent positioning for images captured over time. The device's control processor stores these images, sends reminders for future captures, and generates playback sequences, enabling chronological tracking of teeth whitening progress. Whiteness scores for each analyzed image are calculated using a predetermined algorithm available through the device, making it easier to monitor teeth whitening progress over time without the need for regular dental visits.

### CROSS REFERENCE TO RELATED APPLICATIONS

The present application claims the benefit of, and is a continuation-in-part of, U.S. Non-Provisional patent application Ser. No. 17/825,489, filed May 26, 2022, entitled “Systems and methods to chronologically image orthodontic treatment progress”. This aforementioned application is incorporated herein by reference in its entirety.

### TECHNICAL FIELD

The present invention relates generally to the field of dental health, and more particularly, to systems and methods for analyzing and tracking the progress of teeth whitening treatments. The invention involves the use of portable electronic devices, such as smartphones or tablets, equipped with specific software applications, image capture devices, and display screens. It focuses on innovative techniques for assessing teeth whiteness, monitoring treatment progress over time, and providing users with interactive guidance for capturing consistent and accurate dental images.

### BRIEF SUMMARY OF THE INVENTION

The present invention introduces a comprehensive system and method for teeth whitening analysis and progress tracking, utilizing a portable electronic device. An executable application is designed to guide users through the process of capturing images of their teeth under generally dark or dim ambient lighting conditions. The application further provides instructions for aligning the portable device's camera with the user's teeth and utilizes an alignment guide displayed on the screen for precise positioning.

A key feature of the invention is its capability to evaluate teeth whiteness from captured images in a standardized, repeatable, and quantifiable manner. This is achieved through image processing techniques, including the isolation of pixels within a manually outlined area of the teeth such as the maxillary central incisors, and selective illumination zone rendering on the display divine. The application analyzes obtained images to determine a teeth whiteness score using a predetermined algorithm.

For consistent tracking over time, the invention includes features to capture subsequent images of the user's teeth at approximately the same distance and angulation. One such feature is the superimposing of a previously created manual outline of the teeth on the display screen and instructing the user to align their teeth with this outline for each new image.

Additional functionalities of the invention include employing facial recognition techniques to confirm proper alignment before image capture, selectively rendering illumination zones onto the display screen to illuminate the user's teeth, and generating calendar event reminders for subsequent image captures. The application can also generate a playback image consisting of a series of chronologically captured images, allowing users and dental professionals to visually assess the progress of the teeth whitening treatment and make adjustments as necessary. In some embodiments, the application additionally displays each captured image alongside its corresponding teeth whiteness score, which is calculated and associated with that specific image, enabling a comprehensive assessment of the teeth whitening progress.

Lacking from the prior art are mechanisms to objectively assess teeth whiteness by non-professionals. In a professional setting, cheek retractors, and very expensive cameras are needed to record those images. Even in a professional setting, there is a need to record one or more images at a consistent distance, and angulation, from the subject during the course of treatment. Furthermore, for most patients, regularly visiting dental offices for teeth whitening assessments is impractical both in terms of time and cost.

To address the limitations identified in the prior art, this invention introduces a novel, user-friendly, and efficient method for analyzing and tracking teeth whitening progress, leveraging the widespread availability and advanced capabilities of portable electronic devices.

### BRIEF DESCRIPTION OF THE DRAWINGS

The invention will be readily understood by the following detailed description in conjunction with the accompanying drawings, wherein like reference numerals designate like structural elements, and in which:

FIG. 1 is a block diagram of a system in accordance with an embodiment of the present invention;

FIG. 2 is a flowchart depicting a method of measuring the whiteness of a user's teeth;

FIG. 3 is a flowchart depicting a method of chronologically tracking the progress of a patient's teeth whitening treatment;

FIG. 4 is an environmental view of the manner in which a user begins the process shown in the flowchart depicted in FIG. 2 in accordance with an embodiment;

FIG. 5 illustrates an unaligned alignment guide superimposed on a display screen of a handheld device in accordance with an embodiment;

FIG. 6 illustrates the alignment guide of FIG. 5 in an aligned state;

FIG. 7 illustrates a display screen having rendered onto it one or more illuminating areas in accordance with an embodiment;

FIG. 8 illustrates a user manually outlining the maxillary central incisors in a captured image in accordance with an embodiment; and,

FIG. 9 illustrates the user created manual outline of FIG. 8 superimposed on the display screen in accordance with an embodiment.

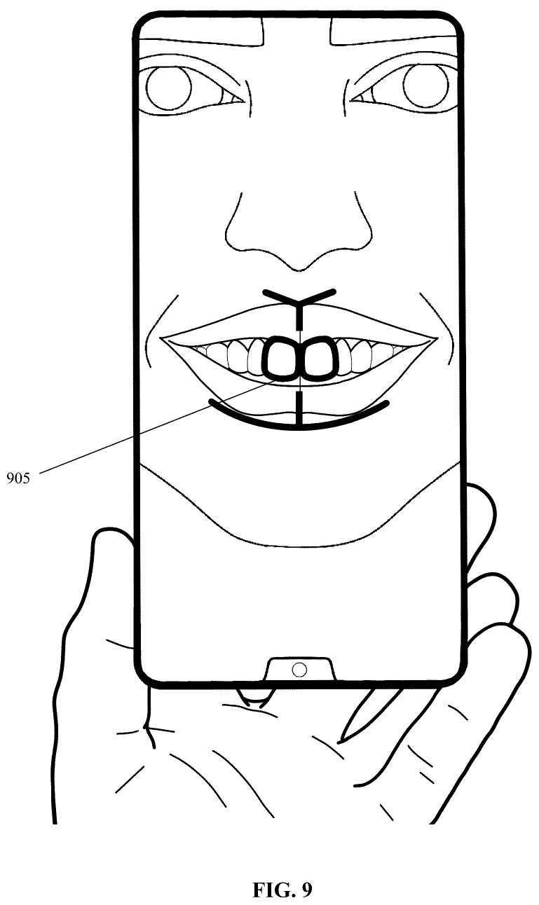

The drawings described herein are for illustration purposes and are not intended to limit the scope of the present subject matter in any way.

### DETAILED DESCRIPTION

In the following description, to better understand the aforementioned purposes, features, and advantages of the present invention, numerous specific details are set forth in order to provide a thorough understanding of the claimed subject matter. It should be noted that these details and examples are provided to merely aid in understanding the descriptions, and they do not, in any way, limit the scope of the present invention. The present invention can also be implemented in other modes different from those described herein and the present invention is not limited to the specific embodiments disclosed below.

The present invention may be a system, a method, and/or a computer program product at any possible technical detail level of integration. The computer program product may include a computer-readable storage medium (or media) having executable computer-readable program instructions thereon for causing a processor to carry out aspects of the present invention.

The specification may refer to “an”, “one” or “some” embodiment(s) in several locations. This does not necessarily imply that each such reference is to the same embodiment(s), or that the feature only applies to a single embodiment. A single feature of different embodiments may also be combined to provide other embodiments.

Furthermore, as used herein, the singular forms “a”, an” and “the” are intended to include the plural forms as well, unless expressly stated otherwise, It will be further understood that the terms “includes”, comprises “, including” and/or “comprising” when used in this specification, specify the presence of stated features, integer steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

Within this disclosure, the terms “portable electronic device”, “electronic device”, “user device”, “computer”, and “smart device” are used interchangeably.

The computer-readable storage medium (“memory”) can be a tangible device that can retain and store instructions for use by an instruction execution device. The computer-readable storage medium may be, without limitation, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including, but not limited to, an object-oriented programming language such as Python, Java, C++, or the like, and procedural programming languages, such as the “C” programming language or similar programming languages. The computer-readable program instructions may execute entirely on the user's device, partly on the user's device, as a stand-alone software package (e.g., an application), partly on the user's device and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus, or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### System Overview

The invention leverages advanced image processing within the application to accurately assess teeth whiteness. The application is designed to function on portable electronic devices with a control processor, an image capture device, typically a camera, and a display screen. It utilizes the device's hardware capabilities along with software algorithms for the desired functionality disclosed herein.

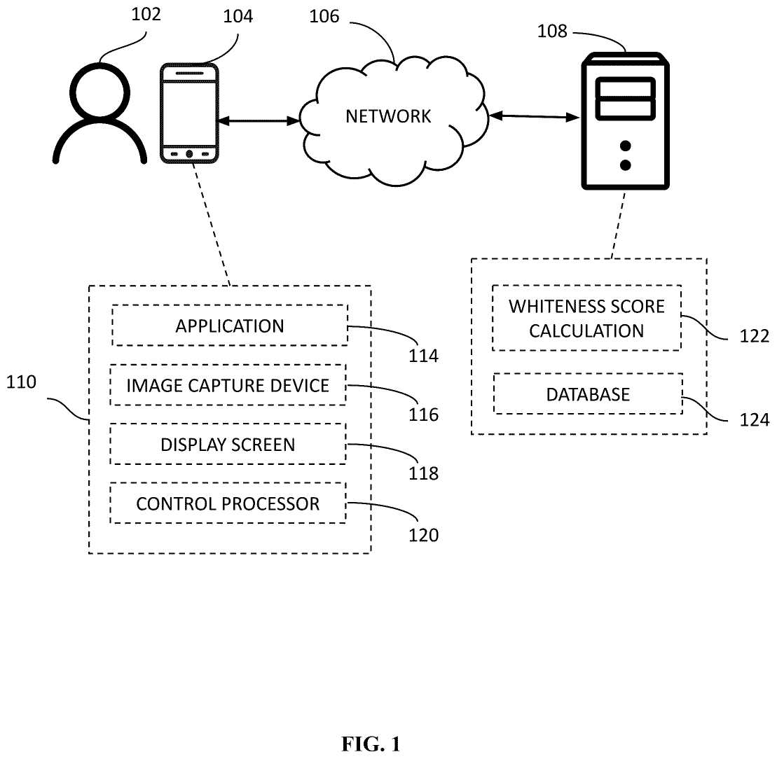

FIG. 1 illustrates a block diagram of a system in accordance with an embodiment of the present invention. A user 102 utilizes a portable electronic device 104, such as a smartphone, to access an application 114 that is communicatively coupled to the device's image capture device 116, display screen 118, and control processor 120. The network 106 is communicatively coupled to both the portable electronic device 104 and a server device 108. The server device is configured to provide whiteness score calculation capabilities 122 and includes a database configured to store user data 124.

In some possible implementations, the system includes a server device that includes one or more processors, memory, and a program for calculating a whiteness score. In some possible implementations, the communicative coupling between the server device and the device is facilitated by the application installed on the device. In some possible implementations, the server device is a physical web server. In some possible implementations, the server device is a cloud server that performs all the same functions of the traditional physical server, including but not limited to, delivering processing power, storage, and applications.

The number and arrangement of devices shown in FIG. 1 is provided as an example. In practice, there may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device may be implemented as multiple, distributed devices. Additionally, or alternatively, a set of devices (e.g., one or more devices) of the aforementioned environment may perform one or more functions described as being performed by another set of devices of the environment.

Facial anatomy presents several amorphous angles and indistinct margins, leading to challenges in consistently positioning the camera for each image in a chronological series. Factors such as varying ambient lighting, the distance between the image capture device and the user, and the orientation of the device relative to the user contribute to these challenges. The techniques disclosed herein are designed to overcome or minimize these variables related to positioning and lighting, thereby ensuring consistent image capture over time. Embodiments of the invention include providing lighting instructions and a source of light, detecting the angulation of the image capture device, offering positional correction guidance, providing pre-made and user created custom facial feature alignment guides, and integrating an image capture mechanism. Additionally, the image capture instructions can be delivered through audio, video, or a combination of both, ensuring a comprehensive mobile solution for capturing consistently positioned images across multiple time intervals.

### Method of Standardized Measurement of User Teeth Whiteness

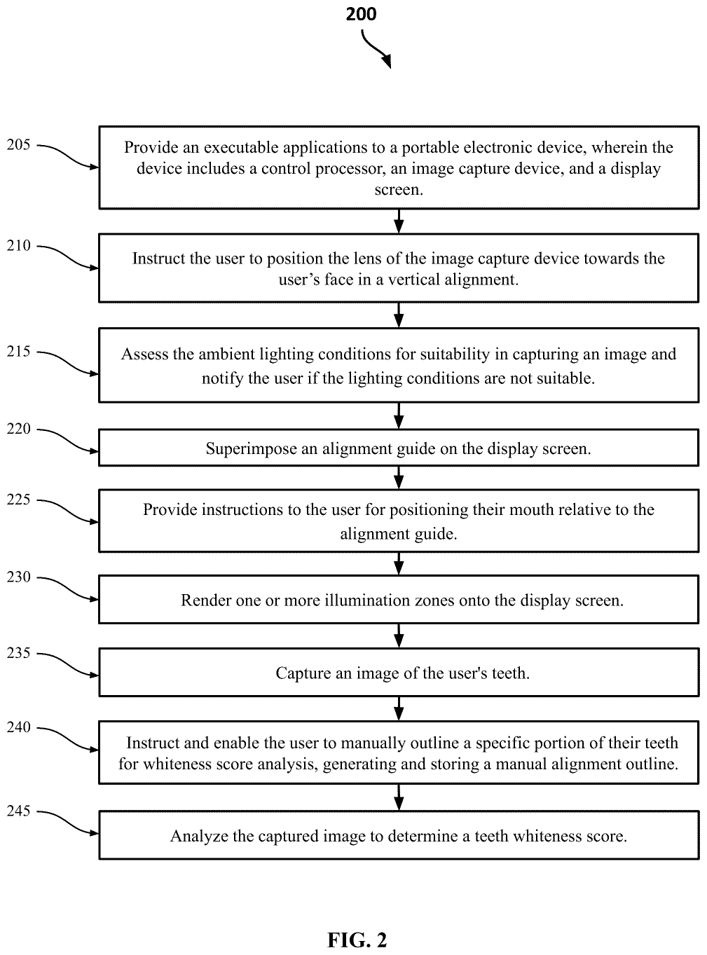

FIG. 2 presents a flowchart depicting a method for measuring the whiteness of a user's teeth. The process begins by providing an executable application to a portable electronic device (referred to as the user device) 305. This user device is equipped with a control processor, an image capture device, and a display screen.

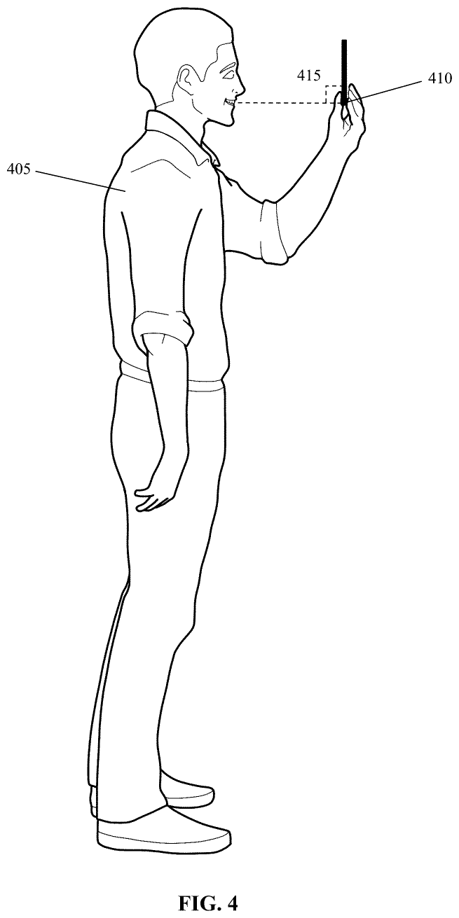

Subsequently, as shown in FIG. 4, the user 405 is instructed 210 to position the lens 410 of the image capture device towards their face in a vertical alignment 415. In the preferred embodiment, the user device utilizes sensors to ensure horizontal alignment and assist the user in this positioning process. This allows the user-side image capture device to detect and display a real time image of the user's face on the display screen.

Following this, the ambient lighting conditions in the user's environment are evaluated for their suitability for capturing an image 315. In the preferred embodiment, the user may choose to capture an image under any lighting condition to calculate a whiteness score. In some embodiments, if the ambient lighting is not sufficiently dim to promote consistent brightness conditions across multiple captures, the user is prompted to decrease the environmental brightness until the lighting conditions are deemed suitable for image capture.

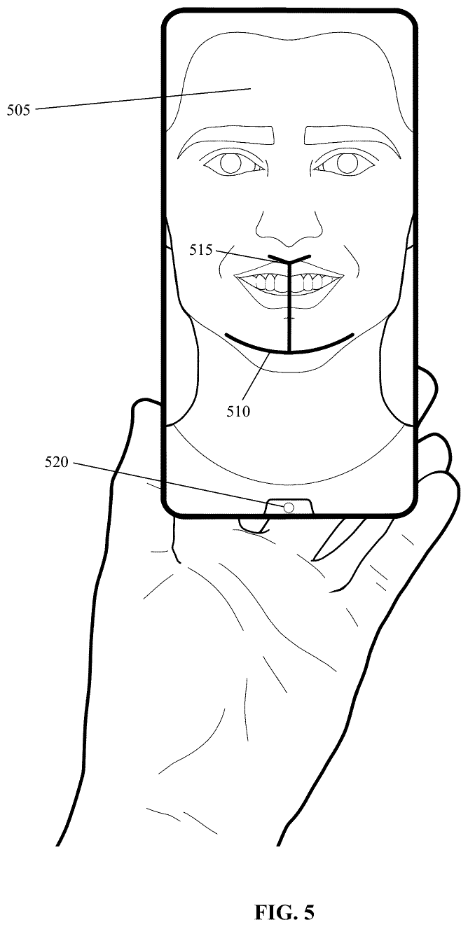

Next, as illustrated in FIG. 5, a default alignment guide 510 is superimposed 320 onto the display screen 505. In this instance, the user's face is too far from the screen, resulting in the correct alignment of their upper vermilion border 515, but misalignment of their lower vermilion border. During this positioning process, the user is provided with instructions 325 on how to position their mouth relative to the alignment guide for proper alignment. In certain embodiments, the application employs real-time image processing to detect this alignment and notifies the user when the alignment is correctly achieved. In the preferred embodiment, the default alignment guide prompts the user to align their central incisor gap with the vertical midline of the guide. Additionally, alignment instructions may include aligning the center of the patient's face, such as the midpoint of the nose or the philtrum, with the guide. In accordance with an embodiment, other facial landmarks, such as specific soft tissue markers (for example, lips, corners of the mouth), can also be utilized for aligning the face with the image capture device.

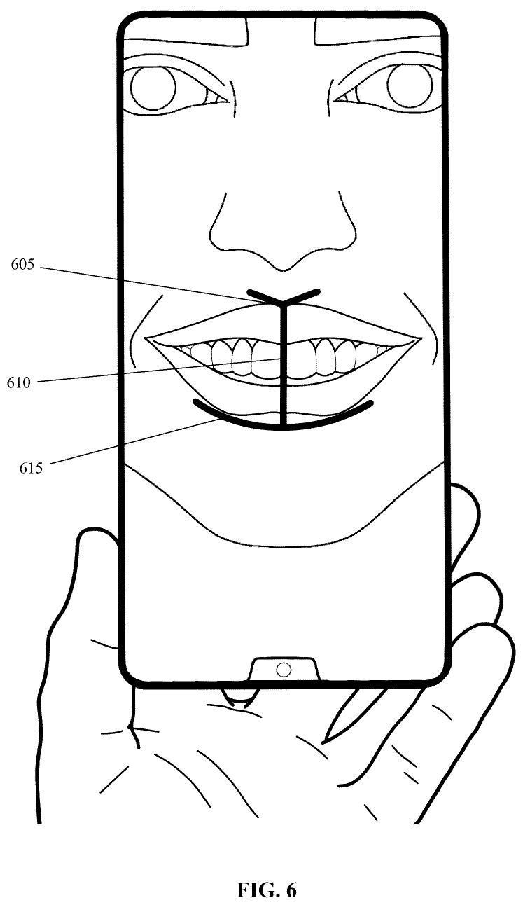

FIG. 6 demonstrates the alignment guide from FIG. 5, now in a state where both the upper and lower vermilion borders 605, 615 are correctly aligned with the default alignment guide 610 and the central incisor gap is aligned with the midline of the guide. Typically, this proper alignment is achieved when the user gradually moves the device closer to their face while ensuring the phone remains vertical throughout.

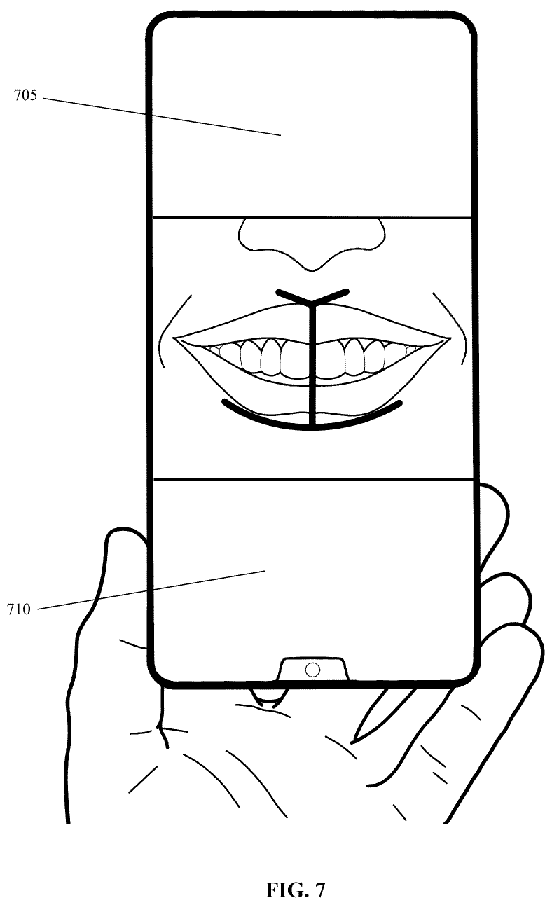

Upon achieving proper alignment, as depicted in FIG. 7, one or more illumination zones 705, 710 are activated on the display screen 230. Subsequently, an image of the user's teeth is captured using the image capture device 235. Preferably, the brightness, color, and size of these illumination zones are adjusted based on the evaluated ambient lighting conditions and specific characteristics of the portable electronic device, such as the phone model. Standardization of teeth whiteness measurements is most effective when the primary source of lighting comes from the illumination zones rendered on the device's screen.

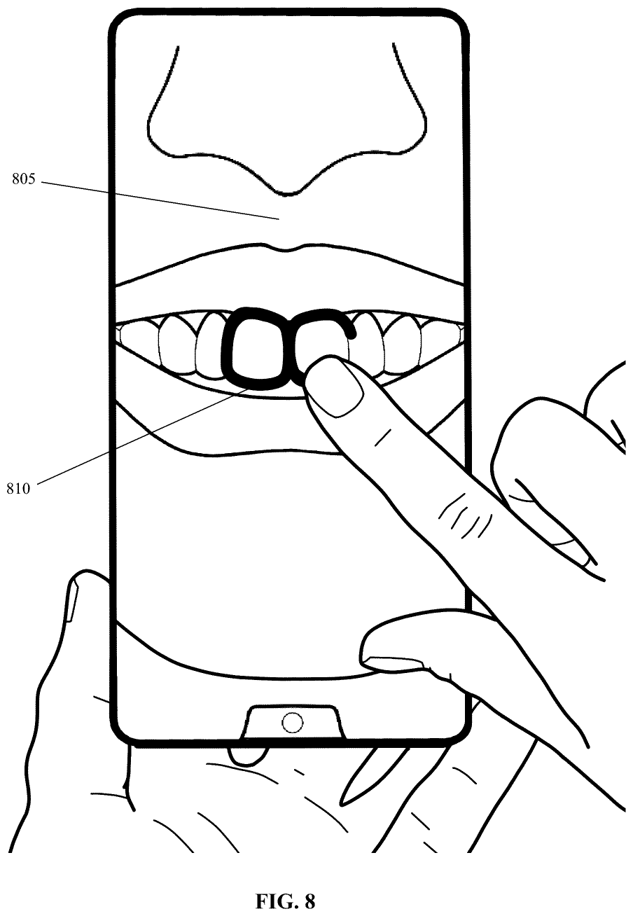

Next, as shown in FIG. 8, the user is guided and enabled to manually outline a specific portion of their teeth 810 on the captured image 805 for whiteness score analysis 240, resulting in the creation and storage of a manual alignment outline. In the preferred embodiment, the application allows the user to use their index finger to trace around one or more desired teeth, to create the manual alignment outline. This process may be repeated to achieve a satisfactory outline. In some embodiments, an offset from the user's finger contact point with the screen is provided to facilitate the manual creation of the alignment outline. This offset addresses potential issues where the user's finger might obscure their view or where the touch input might not precisely align with the visual element being traced, thereby improving the precision and ease of outlining the desired area for the whiteness score analysis.

In some embodiments, the specific portion of the teeth designated for manual outlining is the maxillary central incisors. These teeth are typically the most prominent in most users' smiles and generally exhibit uniform coloration and reflective properties.

Finally, the captured image undergoes analysis to determine the teeth whiteness score 245. In the preferred embodiment, this involves isolating the pixels within the manually outlined area of the teeth. Based on these isolated pixels, a teeth whiteness score is calculated using a predetermined sigmoid function. Typically, a whiteness score ranging from 60 to 90 covers the majority of human teeth whiteness levels, with the average score being between 70 to 75.

In some embodiments, if the lens of the image capture device is situated at the upper end of the user device, the user is instructed to invert the device for image capture before proceeding to step 210. This inversion aids the user in aligning their teeth with the alignment guides, as it allows them to visually ensure their mouth is centrally located on the display screen.

In some embodiments, to maintain consistent distance and angulation in subsequent images of the user's teeth, a manual outline previously created by the user is superimposed on the display screen. The user is then instructed to align their teeth with this outline.

The synergy of an alignment process, which ensures images of the user's teeth are captured from a consistent distance and angle—particularly when employing the user-created manual outline for subsequent photographs—combined with a standardized lighting condition for each picture, leads to uniform and comparable teeth whiteness measurements. This approach enables users to effectively track their teeth whitening progress over time, eliminating the frequent need for professional dental office visits solely for whitening assessments of similar accuracy.

### Chronologically Tracking the Progress of a Patient's Teeth Whitening Treatment

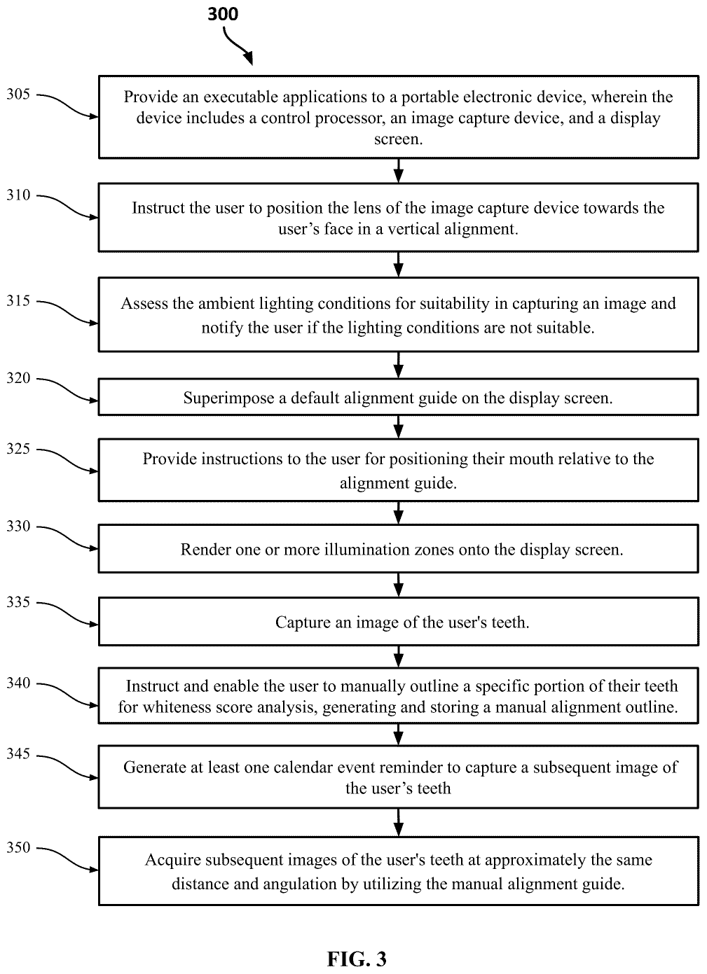

FIG. 3 illustrates a flowchart illustrating a method for chronologically tracking the progress of a patient's teeth whitening treatment using a portable electronic device. This method primarily employs the standardized measurement method for assessing teeth whiteness as detailed in FIG. 2.

In some embodiments, after capturing an image of the user's teeth 335, the image is stored for future reference. Additionally, the system generates at least one calendar event reminder 345 to prompt the user to capture subsequent images of their teeth.

In some embodiments, the time intervals between these calendar event reminders can be set either by the user or a dental professional.

In some embodiments, the control processor of the user device is configured to alert the user based on these calendar event reminders to capture follow-up images of their teeth.

In some embodiments, the control processor is configured to create a playback image comprising a series of captured images of the user's teeth, preferably arranged in chronological order. This feature is particularly useful in the preferred embodiment for visually tracking treatment progress.

In some embodiments, the control processor is configured to evaluate these playback images to identify any instances of treatment relapse or to determine if adjustments to the treatment are necessary.

In some embodiments, the control processor is tasked with analyzing each captured image to compute a teeth whiteness score. This score is then displayed alongside the corresponding image, enabling viewers to see a quantifiable measure of their teeth whitening progress.

In some embodiments, the system provides custom alignment guides to accommodate users with significant anatomical variations. Additionally, in further embodiments, users have the option to disassemble the existing alignment guide silhouettes into individual components and use these fragments to construct a personalized alignment guide.

In some embodiments, the methodology described in FIG. 3 can be used to track patient teeth movements over time. This functionality would be primarily useful for patients undergoing orthodontic treatments that involve teeth movement.

FIG. 3 is a flowchart depicting a method of chronologically tracking the progress of a patient's teeth whitening treatment;

The foregoing disclosure provides illustration and description, but is not intended to be exhaustive or to limit the implementations to the precise form disclosed. Modifications and variations are possible in light of the above disclosure or may be acquired from practice of the implementations.

It will be apparent that systems and/or methods, described herein, may be implemented in different forms of hardware, firmware, or a combination of hardware and software. The actual specialized control hardware or software code used to implement these systems and/or methods is not limited to the implementations. Thus, the operation and behavior of the systems and/or methods were described herein without reference to specific software code-it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

I claim:

1. A method of measuring the whiteness of a user's teeth, the method comprising:
providing an executable application to a portable electronic device, wherein the device includes a control processor, an image capture device, and a display screen;
the executable application enabling the control processor to:
instruct the user to position the lens of the image capture device towards the user's face in a vertical alignment;
assess the ambient lighting conditions for suitability in capturing an image;
superimpose an alignment guide on the display screen;
provide instructions to the user for positioning their mouth relative to the alignment guide;
capture an image of the user's teeth; and,
analyze the captured image to determine a teeth whiteness score.

2. The method of claim 1, wherein if the lens of the image capture device is located at the upper end of the portable electronic device, the method further includes instructing the user to invert the portable electronic device for image capture.

3. The method of claim 2, further including, prior to capturing an image of the user's teeth, rendering one or more illumination zones onto the display screen to illuminate the user's teeth.

4. The method of claim 3, wherein the brightness, color, and/or size of the illumination zones of the display screen are adjusted based on the assessment of the ambient lighting conditions and/or the characteristics of the portable electronic device.

5. The method of claim 1, further including, prior to analyzing the captured image to determine a teeth whiteness score, instructing and enabling the user to manually outline a specific portion of their teeth for analysis.

6. The method of claim 5, wherein the specific portion of the teeth to be manually outlined is specified as the maxillary central incisors.

7. The method of claim 6, wherein, to achieve consistent distance and angulation in subsequent images of the user's teeth, the method includes superimposing the previously created manual outline on the display screen and instructing the user to align their teeth with this outline.

8. The method of claim 7, wherein the analysis of the captured image to determine a teeth whiteness score includes:
isolating the pixels within the manually outlined area of the teeth; and,
calculating the teeth whiteness score based on the isolated pixels using a predetermined sigmoid function.

9. The method of claim 8, wherein the process of isolating the pixels within the manually outlined area of the teeth additionally involves filtering out extraneous elements, such as unintentionally included gum tissue, to enhance the accuracy of the analysis.

10. A non-transitory computer readable medium having stored thereon instructions which when executed by a processor cause the processor to perform a method of chronologically tracking the progress of a patient's teeth whitening treatment, the method comprising:
providing an executable application to a portable electronic device, wherein the device includes a control processor, an image capture device, and a display screen;
the executable application enabling the control processor to:
instruct the user to position the lens of the image capture device towards the user's face in a vertical alignment;
assess the ambient lighting conditions for suitability in capturing an image;
superimpose an alignment guide on the display screen;
provide instructions to the user for positioning their mouth relative to the alignment guide; and,
capture an image of the user's teeth.

11. The computer readable medium of claim 10, wherein the instructions further enable the control processor to instruct the user to invert the portable electronic device for image capture if the lens of the image capture device is located at the upper end of the device.

12. The computer-readable medium of claim 11, wherein the instructions further enable the control processor to, prior to capturing an image of the user's teeth, render one or more illumination zoned onto the display screen to illuminate the user's teeth.

13. The computer-readable medium of claim 12, wherein the instructions further enable the control processor to adjust the brightness, color, and/or size of the one or more illumination zones on the display screen based on the assessment of the ambient lighting conditions and/or the characteristics of the portable electronic device.

14. The computer readable medium of claim 10, wherein the executable instructions further enable the control processor to instruct and facilitate the user to manually outline a specific portion of their teeth to be superimposed on the display screen to achieve consistent distance and angulation in subsequent images of the user's teeth.

15. The computer readable medium of claim 14, wherein the specific portion of the teeth to be manually outlined is the maxillary central incisors.

16. The computer readable medium of claim 15, wherein the executable instructions further enable the control processor to superimpose the previously created manual outline of the teeth on the display screen, and instruct the user to align their teeth with this manual outline, thereby ensuring consistent distance and angulation in subsequent images of the user's teeth.

17. The computer readable medium of claim 10, further including executable instructions to cause the processor to instruct the user to align a vertical midline of the alignment guide with one of their central incisor gap, philtrum center, or facial midline.

18. The computer readable medium of claim 10, further including executable instructions to cause the processor to generate at least one calendar event reminder to capture a subsequent image of the user's teeth.

19. The computer readable medium of claim 18, wherein the interval between calendar event reminders is selected by one of the user and a dental professional.

20. The computer readable medium of claim 18, wherein the executable instructions further enable the control processor to notify the user to obtain a subsequent image of the user's teeth.

21. The computer readable medium of claim 10, wherein the executable instructions further enable the control processor to, before capturing the image of the user's teeth, perform one of the following:
receive a user confirmation indicating that alignment is complete; or
employ a facial recognition technique to automatically determine that alignment is complete.

22. The computer readable medium of claim 10, wherein the executable instructions further enable the control processor to, after capturing an initial image of the user's teeth, acquire subsequent images of the user's teeth at approximately the same distance and angulation by utilizing the same alignment guide as was used for the initial captured image.

23. The computer readable medium of claim 10, wherein the executable instructions further enable the control processor to:
generate a playback image having a series of captured images of the user's teeth; and
determine if a relapse is occurring or if an adjustment to treatment is needed by reviewing the playback image.

24. The computer readable medium of claim 10, wherein the executable instructions further enable the control processor to analyze the captured image to determine a teeth whiteness score.
